(19) United States
(12) Patent Application Publication
(10) Pub. No.: US 2020/0229742 A1
(43) Pub. Date: Jul. 23, 2020

(54) A DIAGNOSTIC SENSOR

(71) Applicants: UNIVERSITY COLLEGE CORK - NATIONAL UNIVERSITY OF IRELAND, CORK, Cork (IE); ALTRATECH LIMITED, Limerick (IE)

(72) Inventors: Brian O'FARRELL, Watergrasshill, County Cork (IE); Ivan O'CONNELL, Mallow, County Cork (IE); Steven DARBY, Spean Bridge, Inverness-shire (GB); Timothy CUMMINS, Cratloe, County Clare (IE)

(73) Assignees: UNIVERSITY COLLEGE CORK - NATIONAL UNIVERSITY OF IRELAND, CORK, Cork (IE); ALTRATECH LIMITED, Limerick (IE)

(21) Appl. No.: 16/648,487

(22) PCT Filed: Sep. 6, 2018
(86) PCT No.: PCT/EP2018/074056

§ 371 Date: Mar. 18, 2020

(30) Foreign Application Priority Data
Sep. 20, 2017 (EP) 17192159.6

(51) Int. Cl. A61B 5/145; G01N 27/327

(52) U.S. Cl. CPC A61B 5/14546; A61B 2562/227; A61B 2562/085; G01N 27/3274

(57) ABSTRACT

A diagnostic sensor device has a semiconductor chip having a distal end physically configured to fit into a power and data socket conforming to a non-proprietary standard, and having exposed pads for engagement with corresponding conductors of such a socket. At its proximal end the chip has at least one sensor for contact with an analyte. The device may be manufactured in a single integrated process to provide a wafer which is diced to provide the individual devices.

### INTRODUCTION

The present invention relates to diagnostics, especially for analytes in the form of liquid droplets.

WO2016/122577 (Hewlett Packard) describes a microfluidic device with microfluidic channels.

JINHONG GUO: “Uric Acid Monitoring with a Smartphone as the Electrochemical Analyzer”, Analytical Chemistry. Vol. 88, no. 24, 20 Dec. 2016 pages 11986-11989, US ISSN: 0003-2700, DOI: 10.1021/acs.analchem.6b04345 describes a polypropylene film with screen-printed carbon electrodes.

### OBJECTIVES

The present invention is directed towards providing a portable diagnostic sensor device which is easy to use for small volume samples such as in droplet form, and/or which allows for rapid assay development, employing multiple connection and detection methods for versatility and increased accuracy.

Another objective is that such a device be suited to applications such as providing detection and quantification testing for beads, DNA, RNA, proteins, and similar analytes.

### SUMMARY OF THE INVENTION

We describe a diagnostic sensor device comprising:
a monolithic semiconductor integrated circuit comprising:
   a distal end physically configured to fit into a connector conforming to a non-proprietary data and power transfer standard, and said distal end having exposed pads for engagement with corresponding conductors of such a connector socket;
   at least one sensor for contact with an analyte, and
   a sensing circuit linked with the or each sensor and said pads.

Because of the monolithic manufacture there are very few steps, merely the well-established semiconductor fabrication steps such as CMOS processing. There is no need for downstream handling and the device may be used immediately after dicing from a wafer.

The device may comprise a layer of hydrophobic material which is etched to form an exposed sensing region for each sensor. Preferably, the hydrophobic material comprises polyimide.

Preferably, the circuit includes converters and digital calibration circuits. Optionally, at least some of the converters and digital calibration circuits are located directly beneath the sensors.

The circuit may comprise capacitive sensors.

The device distal end may be configured to fit into a 12-pin USB connector socket or a 12-pin FFC socket, or for example the sensor device may be configured to mimic a flat flex cable in terms of dimensions.

The sensor regions may be configured to receive and hold sample droplets or beads with different concentrations.

The device may further comprising an interface having engagement pads configured to overlap and engage the pads of the integrated circuit, and the interface comprises contacts for engaging as a male component in a female component of a non-proprietary data and power transfer standard, and optionally the interface has chamfered leading edges. This would provide an assembly of a device which can fit into a non-proprietary socket but may use an interface or “interposer” if it is desired to have sufficient uses that might damage the pads.

We also describe a diagnostic sensor system comprising a sensor device of any embodiment and a host processor programmed to provide power to, and to receive data from, the sensor device via a non-proprietary interface, and to process said data to provide an output.

We also describe a method of manufacturing a sensor device of any embodiment, the method comprising fabricating a wafer with a plurality of semiconductor integrated circuits each forming a sensor device with said configuration, with said pads, with said sensors, and with said circuit, and dicing the wafer to provide the device. It will be appreciated that this is a very simple way of designing and manufacturing a device, requiring only a single fabrication process. There is no need for post-fabrication processes such as packaging and hence little scope for error or damage.

The wafer may be manufactured in an integrated CMOS process in which the pads are deposited and the sensors are formed, and in which the sensors comprise sensor electrodes formed from a top metal layer during fabrication.

Optionally, after completion of wafer processing, the wafer is back-ground to a desired thickness to provide said configuration,

The method may comprise polyimide deposition and etching.

Additional Statements

According to the invention, there is provided a diagnostic sensor device comprising a planar substrate having:
a semiconductor chip with a distal end physically configured to fit into a connector conforming to a non-proprietary data and power transfer standard, and said distal end having exposed pads for engagement with corresponding conductors of such a connector socket;
at least one exposed sensor on the semiconductor chip for contact with an analyte, and
a sensing circuit in the semiconductor chip linked with the or each sensor and said pads.

In one embodiment, the planar substrate is monolithic.

In one embodiment, the planar substrate comprises a layer of hydrophobic material formed on the semiconductor chip and which is etched to form an exposed sensing region on each sensor.

In one embodiment, the hydrophobic material comprises polyimide.

In one embodiment, the circuit includes converters and digital calibration circuits.

In one embodiment, at least some of the converters and digital calibration circuits are located directly beneath the sensors.

In one embodiment, the circuit comprises capacitive sensors.

In one embodiment, the planar substrate distal end is configured to fit into a 12-pin USB connector socket or a 12-pin FFC socket.

In one embodiment, the sensor is configured to mimic a flat flex cable in terms of dimensions.

In one embodiment, the sensor areas are configured to receive and hold sample droplets or beads with different concentrations.

In one embodiment, the device further comprises an interface having engagement pads configured to engage the pads with the planar substrate and the interface overlapping, and the interface comprises contacts for engaging as a male component in a female component of a non-proprietary data and power transfer standard, and optionally the interface has chamfered leading edges.

We also describe a diagnostic sensor system comprising a sensor device of any embodiment and a host processor programmed to receive data from said device via a non-proprietary interface and to process said data to provide an output.

We also describe a method of manufacturing a sensor device, the device comprising:
a semiconductor chip with a distal end physically configured to fit as a male connector into a female connector conforming to a non-proprietary data and power transfer standard, and said distal end having exposed pads for engagement with corresponding conductors of such a female connector; at least one exposed sensor on the semiconductor chip for contact with an analyte, and a sensing circuit in the semiconductor chip linked with the or each sensor and said pads,
the method comprising fabricating a wafer with a plurality of semiconductor chips each forming a sensor device with said configuration, with said pads, with said sensors, and with said circuit, and
dicing the wafer to provide the device.

In one embodiment, the wafer is manufactured in an integrated CMOS process in which the pads are deposited and the sensors are formed, and in which the sensors comprise sensor electrodes formed from a top metal layer during fabrication.

In one embodiment, after completion of wafer processing, the wafer is back-ground to a desired thickness to provide said configuration,

### DETAILED DESCRIPTION OF THE INVENTION

The invention will be more clearly understood from the following description of some embodiments thereof, given by way of example only with reference to the accompanying drawings in which:—

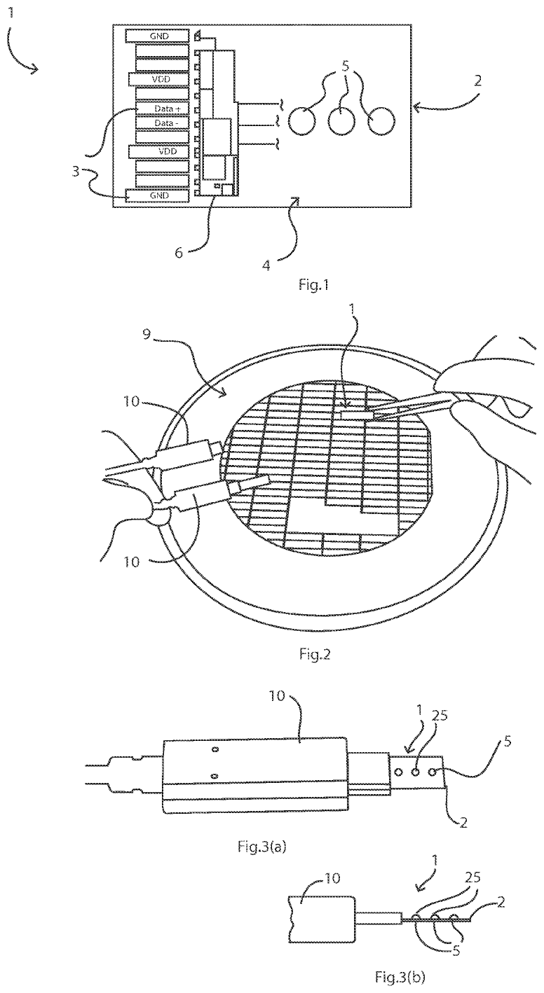

FIG. 1 illustrates a sensor device in the form of a semiconductor monolithic integrated circuit (IC) or “chip”, with positive, negative, and analyte sensors;

FIG. 2 shows a silicon wafer on adhesive film, diced to provide the sensor devices in which the contact, circuit, and sensor components are integrated in a monolithic IC chip;

FIGS. 3(a) and 3(b) show the device inserted into a data and power socket with positive, negative, and analyte droplets placed on the sensors on an exposed portion of the device;

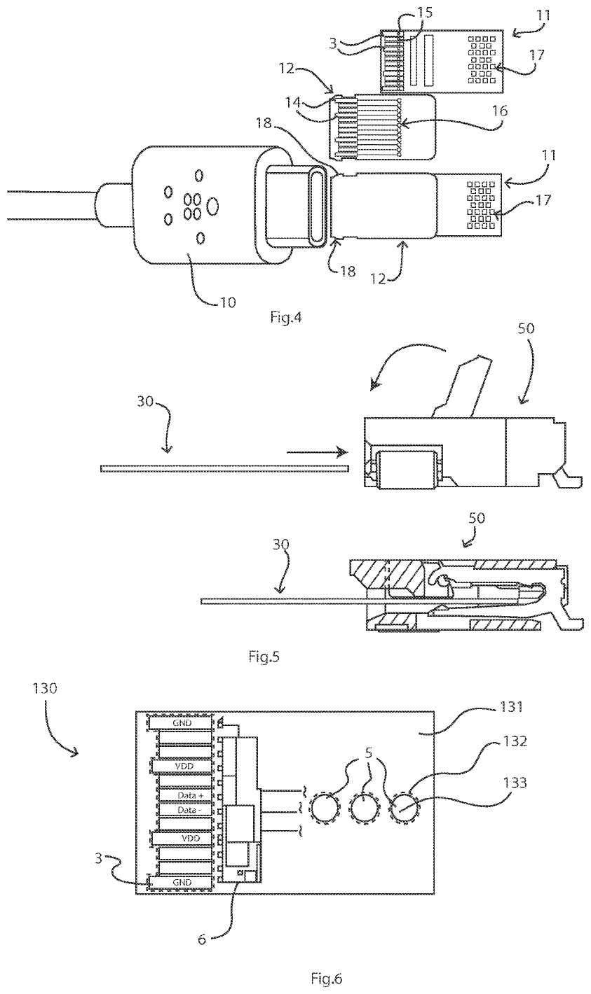

FIG. 4 shows sensor devices with solder balls for attaching to a USB-C mini-adaptor PCB;

FIG. 5 shows a sensor device inserted into a Flat Flex Connector socket; and

FIG. 6 shows a sensor device with a layer of polyimide in which areas are opened.

### DESCRIPTION OF THE EMBODIMENTS

A diagnostic sensor device 1 comprises a silicon planar integrated circuit having a CMOS multi-layer circuit body 2 having a distal end with exposed pads 3 and a proximal end 4 with capacitive sensor electrodes 5. The distal end is physically configured to fit into a power and data socket conforming to a non-proprietary standard, in this case Universal Serial Bus, USB. The proximal end 4 has three sensor regions 5 with capacitive sensor electrodes 5 for detection of an analyte. The multi-layer circuit 6 is alongside and underneath the sensor regions 5 and the contact pads 3. In other embodiments the circuit may be fully located beneath one of the electrodes or sensor regions.

The sensor device 1 is diced from a silicon wafer and it includes in an integrated manner the pads 3, the circuit body (2), the multi-layer circuit 6, and the sensor 5 components. These are monolithically integrated as a semiconductor IC (or “semiconductor chip”). There is no packaging, and the device 1 is planar, as it is diced from a semiconductor wafer in manufacture.

The sensor device 1 distal end has the configuration of a USB-C, being 6.5 mm wide, and having twelve pads 3 with a pad pitch of 0.5 mm, to exactly mimic the male-portion of a USB-C, which typically can be found as a protrusion in the motherboard PCB in laptops and phones, for example in the USB-C slot of the Samsung Galaxy S8 smartphone. The pads 3 are therefore configured to accurately engage the female portion of USB-C, which has 12-spring biased pins, making contact with the 12 pads on the sensor chip. This female part of USB-C is typically found at the end of a USB-C cable (as shown in FIG. 3(a)—which actually has 24 pins, the 12 spring pins being duplicated top and bottom to allow for 180° cable reversal/insertion.

The proximal section 4 three sensor areas 5 are of 1 mm diameter, suitable for receiving a droplet of analyte to be tested. The device's circuit body 2 has a multi-layer circuit 6 linked with both the pads 3 and with the sensors 5 for sensor data processing. The multi-layer circuit 6 comprises CMOS converters, digital calibration circuits, and memory storage. The three capacitive sensors 5 measure and compare the analyte with positive and negative controls, and resolve to femto-Farad and atto-Farad resolution. The circuits may also extend underneath the sensors 5 and/or the pads 3 in a CMOS multi-layer arrangement.

Referring to FIG. 2 the device 1 is cut from a silicon wafer 9 in order to meet the required dimensions. The sensor device 1 may be manufactured in wafer format in an integrated CMOS FAB process with the pads 3 deposited and the sensor regions 5 formed. Wafer fabrication methods of this standard CMOS semiconductor process are known, for example as described in U.S. Pat. No. 5,514,616, which describes transistor source/drain formation, gate deposition, tungsten contacts, CVD oxide deposition, polishing, aluminium metal deposition, and oxy-nitride passivation. The sensor electrodes are typically formed from the top metal layer.

At completion of processing, the wafer is back-ground to a thickness of 0.4 mm for example, to provide a suitable thickness for the sensor device 1. The wafer is then placed on an adhesive film on a ring carrier, which is placed on a wafer-saw machine. This dices the wafer in both X and Y dimensions, leaving individual integrated circuit chips on the film. These can be lifted directly from the film to be ready for use as the devices 1.

Copper or Gold plating of aluminium pads may be employed on CMOS semiconductor processes, as a redistribution layer (RDL) to facilitate solder-bumping or flip-chip assembly. In another embodiment, the interconnect contact pads are plated with copper or gold. This thicker layer facilitates multiple-use insertion and testing.

The arrangement of the sensors and their regions may be chosen at design stage, before the integrated manufacturing process. It will be appreciated that the full sensor device is manufactured in a single process involving CMOS processing, and optionally polyimide deposition and etching. It is therefore monolithic.

FIG. 2 shows how little processing and analysis time is required, as the sensor device 1 is taken directly from a diced wafer on adhesive film 9 straight out of wafer fabrication, and a sample 25 to be tested is placed as shown in FIGS. 3(a) and 3(b) in drops onto the exposed portions 5 and it is inserted into a USB-C connector 10. The connector 10 provides power to the sensor and signals downloaded from a host computer, and the device 1 circuits 2 and 6 upload sensed data which is processed by the host computer to provide immediate results. This is surprisingly simple, rapid and convenient, especially for single-use tests, avoiding the cost and disposal issues of expensive prototype packages.

In another embodiment, referring to FIG. 4, a sensor device 11 with a different sensor region configuration 17 may be manufactured in a manner as described above. The arrangement of the sensors and their regions may be chosen at the design stage, before the integrated manufacturing process. In this case, following RDL plating, the chip connection pads are solder-bumped (15), and the chip is then solder-attached to an adapter or interface (or “interposer”) FR4 PCB 12 with gold-plated pads 14, and a shape and pad-pitch suitable to act as a male part of a USB-C connector. This also enables more robust multiple connect-disconnect uses. The sensor device 11 is shown before and after soldering to the interface 12.

The interface 12 has land-patterns 16 laid out in a pattern matching the solder-bumps 15 in order to receive the solder balls during a flip-and-solder operation. The centre of FIG. 4 shows the interface 12 on the side with the land pattern 16, and the other side of the interface 12 is shown at the bottom of FIG. 4, the sensor device 11 being connected on the hidden side of the interface 12 in this (bottom) view.

Therefore in this embodiment the sensor device 11 is suited to fit directly into a USB socket as for the sensor device 1, but it does not need to do so as the interface 20 is more suited to this function, particularly if multiple insertions are required. The interface 12 has the same width and depth as the sensor device 11 but is more suited to repeated insertion into a socket because it is of PCB material and because it has chamfers and leading tapered side edges 18 to allow easy and accurate insertion into the socket and mating of the twelve gold pads 14 to the twelve spring-loaded pins in connector 10.

In another embodiment, gold-studs form the electrical connections between the sensor device 11 and interposer 12.

Instead of FR4 PCB adaptor material, the sensor device may be attached to a transparent co-polymer adaptor, also configured to fit directly into a standard data and power socket. This facilitates further microfluidic assay integration.

In another embodiment, a sensor device 30 may be configured to fit into a 12-pin FFC socket 50 as shown in FIG. 5, again having twelve pins at 0.5 mm pitch, but in this case the wafer is back-ground to a thickness of 0.3 mm. In this case the device 30 mimics a 0.3 mm FFC flat flex cable in thickness dimensions. This also facilitates rapid single-use or multiple-use testing, in multiple use FFC sockets which are suitable for PCB mounting as shown. The sensor device 30 is inserted in the open socket, and then the cover is closed down, gently pressing the chip pads against the spring pins of the FFC socket. This is another example of a non-proprietary standard data and power connector of which the sensor of the invention forms a male part.

Referring to FIG. 6, in another embodiment, a sensor device 130 has a layer 131 of polyimide, of thickness 4 μm to 10 μm, formed on the wafer during manufacture. The dotted lines 132 show the etch pattern for removing polyimide, from the pads (on left), and from each sensor, see for example the dotted lines 132 surrounding sensor 133. Being hydrophobic, the polyimide acts as a retainer for analyte droplets placed on the sensors, for example by a pipette tip or a droplet processing robot. This facilitates rapid analyte processing and assay development, for example different sensor surface treatments, specific versus non-specific binding experiments, all with real time detection and quantification graphs and readouts on a PC screen via the USB or FFC interface.

A major advantage of the invention is that the sensor device is a planar monolithic integrated circuit without any packaging such as plastics encapsulation nor any fluidic channels. It is manufactured in one process, namely the manufacture of an integrated circuit wafer, in one wafer fabrication facility, with one design and mask-set, with or without a polyimide or other insulation layer, with or without back-grinding to provide for different thicknesses, with or without RDL plating of pads, with or without solder bumps, and with wafers mounted on film and diced, prior to shipment. Upon receipt, the sensor devices are then immediately ready for use. Where many uses are envisaged an interface such as described may be used for inserting into the non-proprietary or standard socket.

The 6.5 mm chip width and 12 pads at 0.5 mm pitch is also particularly convenient, matching the dimensions of both the 12-pin USB-C and 12-pin FFC sockets.

Thus, the expensive upfront investment in one CMOS IC design, layout, and mask set can be leveraged and amortised over many wafer-fabrication options thicknesses, and end-user socket and connection options, facilitating rapid assay development and iteration.

The invention is not limited to the embodiments described but may be varied in construction and detail.

1. A diagnostic sensor device comprising:
a monolithic semiconductor integrated circuit comprising:
a distal end physically configured to fit into a connector conforming to a non-proprietary data and power transfer standard, and said distal end having exposed pads for engagement with corresponding conductors of such a connector socket;
at least one sensor for contact with an analyte, and
a sensing circuit linked with the or each sensor and said pads, wherein the sensing circuit includes converters and digital calibration circuits, at least some of the converters and digital calibration circuits are located directly beneath the sensors, and the circuit comprises capacitive sensors.

2. The diagnostic sensor device as claimed in claim 1, wherein the device comprises a layer of hydrophobic material which is etched to form an exposed sensing region for each sensor.

3. The diagnostic sensor device as claimed in claim 1, wherein the device comprises a layer of hydrophobic material which is etched to form an exposed sensing region for each sensor, and wherein the hydrophobic material comprises polyimide.

4. (canceled)

5. (canceled)

6. (canceled)

7. The diagnostic sensor device as claimed in claim 1, wherein the device distal end is configured to fit into a 12-pin USB connector socket or a 12-pin FFC socket.

8. The diagnostic sensor device as claimed in claim 1, wherein the sensor device is configured to mimic a flat flex cable in terms of dimensions.

9. The diagnostic sensor device as claimed in claim 1, wherein the device comprises a layer of hydrophobic material which is etched to form an exposed sensing region for each sensor, and wherein the sensor regions are configured to receive and hold sample droplets or beads with different concentrations.

10. The diagnostic sensor device as claimed in claim 1, further comprising an interface having engagement pads configured to overlap and engage the pads of the integrated circuit, and the interface comprises contacts for engaging as a male component in a female component of a non-proprietary data and power transfer standard, and optionally the interface has chamfered leading edges.

11. A diagnostic sensor system comprising:
a sensor device comprising:
a monolithic semiconductor integrated circuit comprising:
a distal end physically configured to fit into a connector conforming to a non-proprietary data and power transfer standard, and said distal end having exposed pads for engagement with corresponding conductors of such a connector socket;
at least one sensor for contact with an analyte, and
a sensing circuit linked with the or each sensor and said pads,
wherein:
the sensing circuit includes converters and digital calibration circuits,
at least some of the converters and digital calibration circuits are located directly beneath the sensors, and
the circuit comprises capacitive sensors; and
a host processor programmed to provide power to, and to receive data from, said device via a non-proprietary interface, and to process said data to provide an output.

12. A method of manufacturing a sensor device, the sensor device comprising:
a monolithic semiconductor integrated circuit with a distal end physically configured to fit as a male connector into a female connector conforming to a non-proprietary data and power transfer standard, and said distal end having exposed pads for engagement with corresponding conductors of such a female connector; at least one sensor for contact with an analyte, and a sensing circuit in the semiconductor integrated circuit linked with the or each sensor and said pads, wherein the sensing circuit includes converters and digital calibration circuits, at least some of the converters and digital calibration circuits are located directly beneath the sensors, and the circuit comprises capacitive sensors;
the method comprising fabricating a wafer with a plurality of semiconductor integrated circuits each forming a sensor device with said configuration, with said pads, with said sensors, and with said circuit, and
dicing the wafer to provide the device.

13. The method as claimed in claim 12, wherein the wafer is manufactured in an integrated CMOS process in which the pads are deposited and the sensors are formed, and in which the sensors comprise sensor electrodes formed from a top metal layer during fabrication.

14. The method as claimed in claim 12 wherein, after completion of wafer processing, the wafer is back-ground to a desired thickness to provide said configuration,

15. The method as claimed in claim 12, wherein the wafer is manufactured in an integrated CMOS process in which the pads are deposited and the sensors are formed, and in which the sensors comprise sensor electrodes formed from a top metal layer during fabrication; and wherein the wafer is manufactured in an integrated CMOS process in which the pads are deposited and the sensors are formed, and in which the sensors comprise sensor electrodes formed from a top metal layer during fabrication; and wherein the method comprises polyimide deposition and etching.d
